(19) United States
(12) Patent Application Publication
(10) Pub. No.: US 2023/0352169 A1
(43) Pub. Date: Nov. 2, 2023

(54) SYSTEM AND METHOD FOR AN OPERATING SOFTWARE FOR HUMAN-INFORMATION INTERACTION FOR PATIENT HEALTH

(71) Applicant: Cubismi, Inc., Chevy Chase, MD (US)

(72) Inventors: Scott A. Whitmire, Scottsdale, AZ (US); Moira F. Schieke, Washington, DC (US); John McNeil, Chevy Chase, MD (US)

(21) Appl. No.: 18/309,109

(22) Filed: Apr. 28, 2023

Related U.S. Application Data
(60) Provisional application No. 63/336,660, filed on Apr. 29, 2022.

(51) Int. Cl. G16H 40/67; G16H 10/60

(52) U.S. Cl. CPC G16H 40/67; G16H 10/60

(57) ABSTRACT

The present disclosure is directed to a system and method for augmenting physician expert decision performance and improving healthcare system organization-level decision performance utilizing a joint data space selected from complex patient health data for advanced analytics.

### CROSS-REFERENCE TO RELATED APPLICATIONS

The present application claims priority to U.S. Provisional Patent Application No. 63/336,660, filed Apr. 29, 2022, which is incorporated herein by reference in its entirety.

### BACKGROUND

Human-information interaction (HII) is a multidisciplinary field of study focusing on the ways humans interact with and understand many forms of information and the design of computer technology to support that, in particular, the interaction between humans (the users) and information assisted by computers. While initially concerned with computers, HII has since expanded to cover almost all forms of information processing and presentation design.

Healthcare professional decision performance (DP) is declining in environments with legacy IT systems, legacy interfaces, legacy subjective clinical diagnoses, and exploding volume of data. This combination of trends is leading to the declining quality of decisions, high cognitive effort, high activity times, and high legacy technology costs. Variety, volumes, veracity, and velocity of big data is escalating, as is expert decision complexity. For example, there is an explosion of health data such as the volume of medical images, which has increased approximately 10-fold over the last 20 years. The number of radiologists has only increased 2-fold during the same period, requiring radiologists to read each image at the dangerous rate of approximately 3-4 seconds per image. Fundamental new technologies are needed with systems and methods to improve the ergonomic navigation of patient health big data in order to improve, and in the future augment, expert decision performance.

### SUMMARY

Aspects of the present disclosure relate generally to HII, and more particularly to a system and method for augmenting individual physician expert decision performance and improving healthcare system organization-level decision performance.

Further details of aspects, objects, and advantages of the disclosure are described below in the detailed description, drawings, and claims. Both the foregoing general description and the following detailed description are exemplary and explanatory, and are not intended to be limiting as to the scope of the disclosure. Particular embodiments may include all, some, or none of the components, elements, features, functions, operations, or steps of the embodiments disclosed herein. The subject matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 is an architecture for an extensible software orchestrator (based on a kernel design) that manages a user-directed joint data space (JDS) for multiple types of data processing engines (including an analytics engine, inference engine, and decision intelligence engine) using rule-sets and recipes, in accordance with an illustrative embodiment.

FIG. 2 is a functional flow block diagram of a generic flow of information among the various components of the system and how they are coordinated by a central kernel, in accordance with an illustrative embodiment.

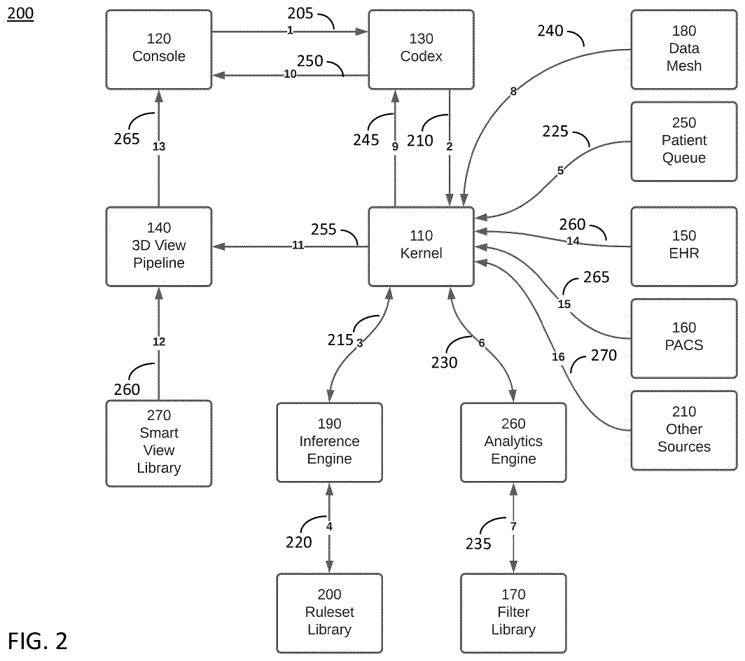

FIG. 3 is a functional flow block diagram of a method for operating on data in the joint data space, in accordance with an illustrative embodiment.

FIG. 4 is a functional flow block diagram of a method for creating a data mesh, in accordance with an illustrative embodiment.

FIG. 5 is a functional flow block diagram for viewing data from the Data Mesh (180), in accordance with an illustrative embodiment.

FIG. 6 is a flowchart of a method for orchestrating human-information interaction (HII) for patient health, in accordance with an illustrative embodiment.

FIG. 7 illustrates a use case, in accordance with some embodiments.

FIG. 8 illustrates a use case, in accordance with some embodiments.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Disclosed herein are embodiments of a system and method for use of a “joint data space” (JDS) selected by users from complex patient health data for advanced analytics to allow ergonomic interaction with big data in order to augment expert decision performance. Embodiments of the method and system describe how a software orchestrator will support and coordinate a system of data processing engines that will operate on the user-selected JDS.

The system and method is designed to allow the expert user to work in synergy with the software orchestrator where both can operate on a “joint data space” (JDS) for a selected patient that is defined by the user at the user console and updated under user control. This design allows the user to shift “frames” of cognitive focus and define a JDS for software orchestrator processing via the console. The orchestrator system uses the user defined JDS to return requested data and information about the data based on various types of analytics back to the user for display on the console.

Each JDS represents a specific sub-set of data selected by the user from the backend patient data mesh (PDM, 180) for display and annotation on the console as needed to select sub-regions of data from the PDM and across various timepoints. Specific nodes or locations in the PDM correspond to anatomical regions and contain all data for all timepoints for each anatomical region. The JDS can contain three-dimensional (3D) or two-dimensional (2D) images and arrays, contain imaging and/or text data, and can be selected from the various types of data, information, or models displayed on the console, such as 2D or 3D images, regions of interest segments of 2D or 3D data, text data displays, or various possible 2D or 3D digital twin models of the patient. The JDS can contain data selected from 3D datacube elements for display of 3D file data in 3D datacubes contained in a 3D medical imaging bioinformatics annotated (“MIBA”) file, 3D precision biomap file, or 3D digital twin files contained within the backend patient data mesh (180) database system. U.S. Pat. Nos. 9,922,433 and 10,347,015 are directed to a method identifying biomarkers using a probability map, U.S. Pat. No. 10,776,963 is directed to a method for forming a super-resolution biomarker map image, U.S. Pat. No. 11,213,220 is directed to a method for determining in vivo tissue biomarker characteristics using multiparameter MM matrix creation and big data analytics, and U.S. Pat. No. 11,232,853 is directed to a method for creating, querying, and displaying a MIBA master file, which are all incorporated herein by reference. The JDS data can include all vector data across all timepoints for any number (1, 2, 4, 600, 805, 1000, 100,000, etc.) of corresponding vector points (for example, including a vector biomarker output for “is the voxel normal?”) located in a single voxel of a PDM 3D file, such as a 3D MIBA file, 3D precision biomap, or 3D digital twin for display on the console. Alternately, the JDS could contain a single vector point for a single voxel (for example, a vector biomarker output for “is the voxel normal?”) of a PDM 3D file, such as a 3D MIBA file, 3D precision biomap, or 3D digital twin for display on the console. Each piece of data in the JDS carries an identification tag (ID) corresponding to a specific node and/or location in the Patient Data Mesh (PDM, 180).

The full IDS data and/or ID for each piece of data in the IDS may be located in server side (e.g., the kernel such as memory in or coupled to the kernel). The full (or subset) of IDS data and/or ID for each piece of data may be shared with the client. The full (or subset) of IDS data and/or ID for each piece of data can be updated on the server side and the client can request the update be shared. The kernel may determine the patient, time point, body site, and other user selections and key data parameters for use in orchestrator recipes based on the conditions in the IDS (e.g., stored in the memory). The engines receive the data in the form of parameters passed as part of the recipe call into the respective engine. For example, the recipe options shows the one parameter (e.g., “name” with the value “Scott”) to the “printName” recipe. An executed orchestrator recipe would print the name, “Scott.” Option key/value pairs may be separated by commas in that list. Option key/value pairs may be sent as a package in an API or procedural call.

Each IDS instance can include a log of status of requests and responses corresponding to the body location for the patient. The coordination between the user IDS selections and backend database and analytics system is managed by the kernel orchestrator.

As the user shifts their cognitive “frame” and selects a new IDS, the software orchestrator generates a new instance for a secondary and new IDS which can be an entirely new anatomical location or series of anatomical locations, or a new subset of data for the same anatomical location, or a set of clinical data with no specific anatomical correlate. The software orchestrator begins a parallel process for the new instance that runs in parallel with first IDS instance. All work in process will continue, with the resulting information passed to the kernel which will send updates to the console in parallel with any new processing.

A central kernel can serve as the primary communicator and translator for the user and to direct the system in taking actions on the joint data space (JDS). The kernel can interpret, translate, and act on user commands and questions using the IDS and triggers numerous potential specific rules, recipes, and algorithms. The kernel can conduct or navigate traffic between a frontend and a backend databases and data processing engines based on expert user commands and specifics system of codecs. The backend can include a core Data Mesh technology. Nodes in the Patient Data Mesh (PDM, 180) can “point” (using data ID tags and database code) to formatted 3D files including the 3D MIBA file, 3D precision biomap, and 3D digital twin. The Data Mesh can include analytics data provided by the backend system analytics or third party algorithms in the analytics engine that can be added to the Data Mesh upon user command, or “point” to analytics data in separate databases, such as 3D MIBA file, 3D precision biomap, and 3D digital twin. For example, a specific digital twin can be created based on analytics on top of selected 3D database file, or analytics could have happened before and updated previously to the 3D MIBA file for immediate display.

The kernel can be designed for “conversation” with the expert user through a hierarchical model that utilizes the JDS and the decision stage of the user (entered via a UI element for the user to denote decision stage) as the primary inputs for the kernel. The kernel can be designed for the user to ask broad questions such as “what's my most likely differential diagnosis?” and ask questions back to the user to further refine the question and assure execution of the correct specific ruleset.

The user selects the JDS at the console that are translated by codecs for the user to view and select the specific data for each JDS. The kernel manages backend novel JDS selection and orchestration of multiple data processing engines, creating multiple novel user-directed data processing capabilities.

One novel JDS capability is to query and retrieve raw data for a single patient user-defined JDS for a selected anatomical region of interest (ROI) for all available timepoints (UC 1) from the Patient Data Mesh (PDM) (180) for display on the console. Another novel JDS capability is to select a single image or 3D MIBA file datacube within the user-selected JDS for a single patient (UC 2) from the PDM for display on the console.

Another novel analytics capability is to select analytics/categorization question for single patient (UC 4) for a specific user defined JDS for data processing by the analytics engine by executing recipes. For example, “what is my probability that the regions of anatomy contained within this JDS represents cancer?”

Another novel analytics capability is for the user to allow the backend inference engine to execute rule-sets to seamlessly make predictions about data in the defined user JDS, such as “what's the most likely differential diagnosis?” (UC 5) and display these guesses at the user console. Another novel analytics capability is that user selection can turn specific inference engine commands acting seamlessly on IDS data on or off for single patient (UC 6).

Another novel analytics capability is for the user to request a decision intelligence predictive question from the DI engine (connected to a population database of PDM) for the given IDS by executing a recipe, such as “what are the potential patient complications versus benefits from a decision to biopsy versus recommend 6 month follow-up imaging?” (UC 7). Another novel analytics capability is to allow a combination of the analytics engine, decision intelligence engine, and inference engines to act on the IDS for various specific analytics requests as needed (UC 8) by following a defined set of specific multi-engine rule-sets and recipes.

Another novel analytics capability is to allow the user, when needed, to select a pre-trained recipe library for the analytics engine, or to select population database data for a given analytics question to run a new classification of prediction for a given IDS (UC 9). Another novel analytics capability is to ergonomically re-review key IDS data or analytics results held in IDS system memory for single patient (UC 10) during a given user session and choose to save the analytics results to the Patient Data Mesh (PDM) by the user for later retrieval. In some cases, user results or information may return days after the initial request, such as a pathology report from a given biopsy, and can be added to the PDM upon user command.

Another novel analytics capability is to that user can turn automated logging of IDS data to the Patient Data Mesh on or off for single patient (UC 11) during a given session.

Another novel IDS capability is to ergonomically connect multiple users via separate consoles to a IDS display (UC 13) during a given user session for a single or multiple selected user defined IDS. Another IDS capability is to allow multiple users ergonomically interact with data in the IDS for a single patient where the IDS may be raw data, such as a single image, and/or a single voxel from a 3D data vector from secondary 3D file databases in the PDM. One novel analytics capability is to ergonomically interact with data in the PDM for a single patient across a full patient journey (a series of IDS at various timepoints) to analyze decision performance and patient outcomes (UC 14) using decision intelligence console for a single patient, a group of patients, or a population of patients.

Another novel analytics capability is to ergonomically interact with data in a given JDS at a Decision Intelligence Console for a population of patients to analyze decision performance. Another novel analytics capability is to ergonomically seek analytics related to specific question/hypothesis for given JDS for a population of patients (UC 15) via use of an inference engine. Another novel JDS capability is to ergonomically interact with data in joint data space (JDS) for a single patient across a full patient journey to analyze the quality of patient outcome using decision intelligence analytics (UC 16).

Another novel analytics capability is to ergonomically interact with data in a JDS for a population of patients to analyze the quality of patient outcomes using decision intelligence analytics (UC 17).

Another novel JDS capability is to display all vector data across all timepoints for any number (1, 2, 4, 600, 805, 1000, 100,000, etc.) of corresponding vector points (for example, including a vector output for “is the voxel normal?”) located in a single voxel of a PDM 3D file, such as a 3D MIBA file, 3D precision biomap, or 3D digital twin for display on the console.

Advantageously, embodiments of the system and method are extensible and flexible in that they can expand using multiple libraries, or any number of models (e.g., neural networks) or algorithms used to make decisions and diagnoses associated with a patient journey for a single or combination of JDS. Different models and algorithms can be added for different anatomical features, different diseases, etc. Each model can have a different set of rules. Embodiments of the system and method can expand the number of models without changing any code, script, software, firmware, etc.

A benefit is that embodiments of the system and method can provide varying levels of granularity of the data. A feature that is analyzed can be an entire image. Likewise, the analyzed feature can be identified as a single voxel of the image or a vector with many types of data mapped for a single voxel in the 3D files in the PDM, such as a single 3D MIBA file voxel. The voxel may be associated with an area of a patient's anatomy.

Of the various novel features and benefits listed above and throughout this specification, not every feature listed herein need be present in the system. Various embodiments of the system as described herein may include any one or more of the features alone or in combination.

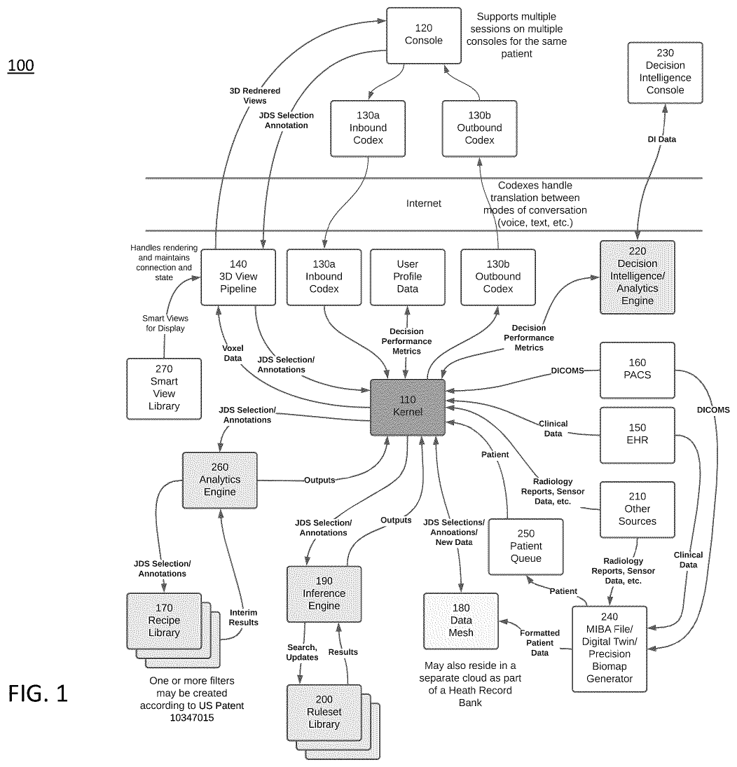

FIG. 1 is an architecture for an extensible orchestrator 100, in accordance with an illustrative embodiment. In some embodiments, the orchestrator software 100 includes a kernel 110 (structurally similar to a management kernel, Linux kernel, Unix kernel, etc.), a console 120, one or more codecs 130 for each direction of communication, a 3D View Pipeline 140 that makes use of a “smart views” library 270, an analytics engine 260 that makes use of one or more recipes and other pre-defined analytics training algorithms 170 or directs use of secondary analytics engines, a data store organized to support the process required for the platform Data Mesh 180 (which contains many individual Data Mesh's for each patient), an inference engine 190 (e.g., hypothesis engine) that makes use of one or more rulesets, which may include decision and disease models, 200, an interface to one or more Electronic Health Records (EHR) 150, an interface to one or more Picture Archival and Communication Systems (PACS) 160, other backend databases such as CRM, radiology reports, etc., a generator for the 3D MIBA File/3D Digital Twin/3D Precision Biomap 240 (which can also be referred to as a PDM generator), a patient queue 250, a Decision Intelligence analytics engine 220 (e.g., a decision intelligence analytics engine, decision intelligence engine), and an organization Decision Intelligence console 230.

The kernel 110 can manage state and communications for one or more user sessions accessing a patient simultaneously, which can allow multiple users on separate consoles 120 to collaborate. The kernel 110 can be coupled to each of the components of the operating software 100 (e.g., the analytics engine 260, the hypothesis engine 190, the decision intelligence analytics engine 220, etc.). The kernel 110 can be coupled to each of the components directly, indirectly, through an interface, etc. The kernel 110 can direct traffic and serve as a buffer between components of the operating software 100. The kernel 110 can manage the communications between multiple sessions (e.g., updating all users when one user does something). The kernel 110 may keep track of the current state of the operating software 100 and the JDS instances. The kernel 110 can allow fast (e.g., millisecond), intelligent, cloud-based, human-computer interaction (HCI) between a user expert and the operating system and software 100 components.

The operating software 100 can support multiple sessions on multiple consoles such as the console 120 for the same patient.

In some embodiments, the codex pairs 130a and 130b encode and decode various communications modes between that used at the console 120 and that used in the kernel 110. Communications modes may include voice conversation in one or both directions, textual conversation in one or both directions, or point-and-click with one or more pointing devices, including but not limited to a mouse, stylus, pencil, or finger gestures.

The 3D view pipeline 140 can manage and control the transmission of 2D and 3D images and 3D datacube voxelwise data between the kernel 110 and the console 120 in both directions. In some embodiments, the 3D view pipeline 140 manages and controls the transmission of image data and 3D datacube voxelwise data in order to adjust the quantity and speed of data transmitted to fit the capacity of the network connection and the computer running the console.

In some embodiments, Electronic health record systems (EHR) 150, Picture Archival and Communication Systems (PACS) 160, and devices and sensors connected directly to the network (IoT devices) or other sources 210, are external data providers. The operating software 100 can include interfaces to interface with specific systems and devices. These interfaces can be installation dependent and can provide a common application programming interface (API) to the kernel 110.

The analytics engine 260 can direct a network of networks by applying pre-trained models for various types of data using specific recipes. The image or 3D datacube filters or other pre-trained analytics algorithms recipes 170 may be neural networks trained and optimized to detect a specific feature in data within the JDS. The filters or other analytics algorithms 170 may implement a specific data analysis algorithm such as noise reduction or edge detection. In some embodiments, a recipe acting on many defined JDS may apply an implementation of a moving window (“MW”) algorithms to create biomarker maps or a super-resolution biomarker maps using a datacube. Details of the biomarker maps and super-resolution biomarker maps are described in the U.S. Pat. No. 10,347,015, which is incorporated herein by reference. Other incorporations by reference are disclosed above. The specific analytics recipe or algorithms used are selected by the expert user and can depend on the situation for the patient, available data needed for a given filter or algorithm, the anatomical structure under examination, and other guidance from the expert human user via the console 120. Recipes or algorithms created using different means may be used together in sequence and are stored in a library.

A Patient Data Mesh 180 (containing many individual patient Data Mesh's) can be a graph database which points to secondary databases, such as for 3D MBA files, 3D precision biomaps, or 3D digital twins, as well as other databases holding primary raw data such as the PACS, EHR, etc. as described above, with coordinated ID tags for each piece of data. The Data Mesh 180 and individual Patient Data Mesh's may make use of a variety of known data organizing structures, including relational, graph, semantic, or any other form, or a combination of database types.

In some embodiments, the analytics engine 260 uses knowledge of the anatomical structure identified in the PDM and corresponding user-selected IDS and other input from the expert human user via the console 120 for the kernel 110 to execute an ordered set of analytics algorithms or recipes from the available recipes 170 to apply to an image or 3D datacube or other combinations of data selected for the IDS. The analytics engine 260 can apply pre-trained models for various types of data, including pre-trained filters for single images, while remaining in the scope of the present disclosure. The final analytics output can be converted into findings interpretable by the kernel and which are communicated to the kernel 110.

Recipes 170 can include pre-trained filters to define anatomy segmentations associated with PDMs. For example, there may be a pre-trained filter to find the liver in a PDM image containing the tag, “abdomen,” another to find the lungs in a “chest” image, and the like. Each filter can be registered to the image in order to remove image data for other anatomical features, leaving only the patient's anatomical feature associated with the recipe (e.g., liver) or generate an annotation of the liver segmentation on the source image, and the recipe will generate an ID tag for the identified liver. After anatomy segmentation (e.g., image shows it has a liver) the anatomical feature annotation can be used to map or “point” the image to a node on the graph database PDM (180). In this way, the orchestrator system will allow for more granular anatomical data organization over time. In this example, the source PDM “abdomen” image would receive a more granular tag of “liver,” an organ contained in the liver.

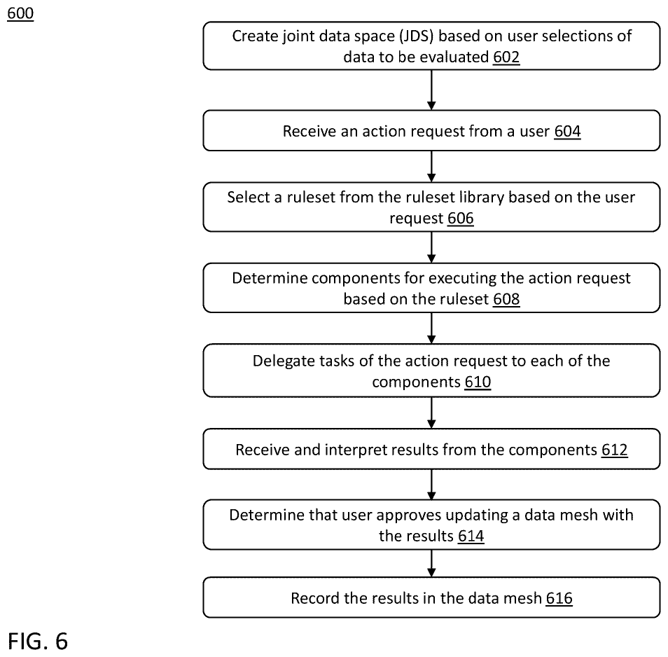

FIG. 6 is a flowchart of a method 600 for orchestrating human-information interaction (HII) for patient health, in accordance with an illustrative embodiment. The method 600 may be implemented using, or performed by one or more of the operating software 100, one or more components of the operating software 100, or a processor associated with the operating software 100 or one or more of the components. The method 600 may be performed by the kernel 110. Additional, fewer, or different operations may be performed in the method 600 depending on the embodiment. Additionally, or alternatively, two or more of the operations or embodiments of the method 600 may be performed in parallel. Operations or embodiments of the method 600 may be combined with one or more operations or embodiments of one or more of the methods 200-500.

At operation 602, the kernel 110 creates or modifies a joint data space (JDS) based on user selections of data to be evaluated.

In some embodiments, the kernel 110 determines a current state of a joint data space (JDS). In some embodiments, the kernel 110 determines a current state of the JDS by determining whether the JDS has been established. In some embodiments, in response to determining that the current state of the JDS is that the JDS has not been established, the kernel 110 loads or maps a patient data mesh 180 for the specified patient into the JDS to generate a JDS for the specified patient. The data mesh 180 can “point” (using data ID tags and database code) to analytics data in separate databases, such as 3D MBA file, 3D precision biomap, and 3D digital twin.

At operation 604, the kernel 110 receives an action request (e.g., a question) from the user. Examples of a request include “what is my probability that this JDS indicates cancer?” or “what is my most likely differential diagnosis?” In some embodiments, the kernel 110 receives the question or request from the console 120 via a codex 130. In some embodiments, the kernel 110 determines a current state of the JDS for a patient specified in a question or request sent to the kernel 110 by a user.

At operation 606, the kernel 110 selects a ruleset. In some embodiments, the kernel 110 selects the ruleset in response to determining that the current state of the JDS is that the JDS has been established, or in response to loading the patient data mesh 180 into the JDS. The kernel 110 can select the ruleset to execute based on criteria. Criteria for selecting the ruleset can include one or more of: (1) a body site that was selected, (2) a decision stage, (3) a type of question is being asked, or (4) a current state of the JDS. The type of question being asked can include one or more of categorization, filtering, prediction, patient prediction, user prediction, or digital twin prediction.

At operation 608, the kernel 110 determines components for executing the action request based on the ruleset. In some embodiments, the ruleset prescribes the components, and the kernel 110 identifies the components prescribed by the ruleset.

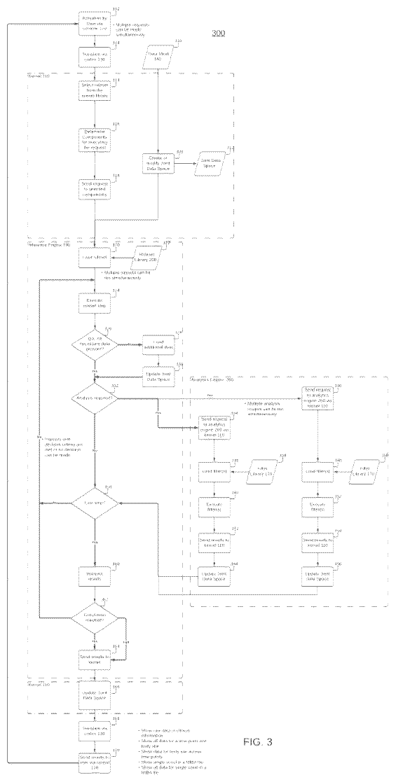

At operation 610, the kernel 110 delegates tasks/portions of the action request to each of the components. The components may include an inference engine 190 and an analytics engine 260. In alternative embodiments, the inference engine 190 and/or the analytics engine 260 may be replaced or supplemented by an intelligence agent which may include one or more computing components that utilize an artificial intelligence and/or machine learning algorithm to adapts and evolves or learns based on usage of the system. For example, the intelligence agent may utilize a neural network or other artificial intelligence system to receive inputs and take actions and/or make responsive decisions to the inputs such that the actions and responsive decision making process and ultimate decisions evolve over the lifetime of the system. An exemplary flow with such components is illustrated in FIG. 3.

At operation 612, the kernel 110 receives results from one or more of the components. Examples of results include raw data or clinical information; all data for a time point and body site; data for body site across time points; single voxel in a MIBA file, biomap, digital twin, 3D file, or graph database; data for a single voxel in a MIBA file, biomap, digital twin, 3D file, or graph database; a differential diagnosis; a probability; etc.

At operation 614, the kernel 110 determines that the user approves updating a data mesh with the results. In some embodiments, the kernel 110 asks the user, via the console, if the data mesh should be updated or the analytics results discarded. The kernel 110 may indicate that the results are available (e.g., via the console 120). In some embodiments, the kernel 110 interrupts a user. In some embodiments, the kernel 110 updates a status board (e.g., without interrupting the user). In some embodiments, the kernel 110 sends the results to the console 120, via the codex 130. In some embodiments, the codex 130 translates the results. In some embodiments, the console 120 provides the results to the user.

At operation 616, the kernel 110 records the results in one or more of a data mesh, a graph database, a MIBA file, a biomap, or a digital twin. The kernel 110 may record the results in response to determining that the user has approved updating the data mesh. In some embodiments, the kernel 110 updates the JDS with the results. The JDS can include a pointer to a portion of the graph database, MIBA file, biomap, or digital twin. In some embodiments, when the JDS is updated, the results are automatically recorded in the graph database, MIBA file, biomap, or digital twin. In some embodiments, the kernel 110 updates the data tags and database code used to “point” the data mesh 180 to analytics data in separate databases, such as 3D MIBA file, 3D precision biomap, and 3D digital twin.

FIG. 3 is a functional flow block diagram of a method 300 for operating on data in the joint data space, in accordance with an illustrative embodiment. The method 300 may be implemented using, or performed by one or more of the operating software 100, one or more components of the operating software 100, or a processor associated with the operating software 100 or one or more of the components. Additional, fewer, or different operations may be performed in the method 400 depending on the embodiment. Additionally, or alternatively, two or more of the operations or embodiments of the method 300 may be performed in parallel. Operations or embodiments of the method 300 may be combined with one or more operations or embodiments of the method 200.

In some embodiments, prior to the operations in FIG. 3, the user selects a patient, at which time the kernel 110 can command the inference engine 190 to execute the ruleset from the ruleset library 200 to create the graph database and/or MIBA file from the raw data. The MIBA file can use standard registrations to create datacubes or use precision biomap methods. This processing can occur in the background on the backend. In some cases, the graph database and 3D MIBA files would already exist.

In some embodiments, FIG. 3 depicts a flow of a session in which the kernel 110, the inference engine 190, and the analytics engine 260 together operate on data in the joint data space to answer a question posed by the user via the console 120. The question may be one of: (1) categorization (“Does the selected region contain normal or abnormal tissue?”), (2) filtering (“Exclude the skull from this image view”), or (3) prediction, which may take one of: (a) patient (“What is the likely prognosis for this patient?”), (2) user (“Which image or data will the user want to see next?”), or (3) digital twin (“How will this tumor respond to a given course of treatment?”).

At operation 302, a user, via the console 120, activates the session. The user may activate by asking a question, sending a request, selecting an input, changing data, and the like. Multiple requests can be made simultaneously. The question may be one of categorization, filtering, or prediction. The type of prediction may be one of patient prediction, user prediction, or digital twin prediction. At operation 304, the codex 130 translates the request.

In some embodiments, the kernel 110 determines a current state of the joint data space (JDS), for example, for a patient specified in the question of 302. In some embodiments, the kernel 110 determines a current state of the JDS by determining whether the JDS for the selected anatomical location has been established. In some embodiments, the kernel 110 determines that the JDS has been established if the kernel 110 determines that the corresponding data in the data mesh for the specified patient has been loaded. In some embodiments, the kernel 110 determines that the JDS has not been established if the kernel 110 determines the corresponding data for the selected anatomical location has been loaded into the data mesh for the specified patient.

At operation 308, the kernel 110 loads the data from the data mesh for the specified patient, shown as input 310, into the JDS to generate JDS 312 for the specified patient and anatomical location. Operation 308 may be in response to determining that the current state of the JDS is that the JDS does not exist.

At operation 314, the kernel 110 selects a ruleset, e.g., in response to determining that the current state of the JDS is that the JDS has been established, or in response to loading the patient data into the JDS. The kernel 110 determines the ruleset to execute based on the current context (e.g., current state of the joint data space), and the question posed by the user (e.g., the request).

Criteria for selecting the ruleset can include (1) which body site was selected, (2) what is the current decision stage, or (3) what type of question is being asked. Rulesets can specialize in categorization, filtering, or predicting, and by body site within a question type. This may be implemented as hierarchies of rulesets and could be very simple to very complex. The type of question can be determined by the action taken by the user, or by analysis of a text or voice command (e.g., using some form of natural language processing). Examples of questions can be “provide a differential diagnosis for this lung,” “who do I need to consult with?”, “remove the skull from this brain MRI,” or “what is the likely response of this tumor to this course of treatment?”

In some embodiments, the kernel 110 can determine whether user approves of the ruleset selection. In response to determining that the user does not approve of the ruleset selection, the method 300 returns to operation 314 and the kernel 110 chooses another ruleset. At operation 316, the kernel 110 determines components for executing the request from operation 302. At operation 318, the kernel 110 sends a request to the inference engine 190. The request can be the request of operation 302. The request can be a request to execute a ruleset. The request can include the ruleset. Operations 316 and 318 may be in response to determining that the user approves of the ruleset selection.

At operation 320, the inference engine loads the ruleset. Rulesets that govern the flow of an analysis, including disease and decision models, are stored in the ruleset library 200, as shown in input 322. Any model that can be represented as a ruleset or procedure can be stored in the library, extending the scope of the operating software 100 well beyond radiology or even medicine.

At operation 324, the inference engine 190 executes the ruleset. As the inference engine 190 executes the rule set, any requests for processes or additional data can be made in parallel with the results being updated to the joint data space by the kernel. Many such analyses can be done simultaneously. At operation 326, the inference engine 190 determines whether all necessary data is present to execute a step of the ruleset. In response to the inference engine 190 determining that not all of the necessary data is present, at operation 328, the inference engine 190 or the kernel 110 loads additional data into the graph database, and/or 3D MIBA file, and/or IDS as needed. For example, additional data can be added based on a user request for additional data. At operation 330, the kernel updates the joint data space. In response to determining that all necessary data is present, or in response to updating the joint data space, at operation 332, the inference engine 190 determines whether analysis is required to execute the ruleset.

At operation 334, the analytics engine 260 receives a request from either the interface engine 190 or the kernel 110. The request can be the request of operation 302. The request can be a request to execute filters. The request can include a request to apply a recipe to detect a specific feature in data within the IDS. Recipes for analytics are stored in the analytics engine 260 in a library (creates extensibility).

At operation 336, the analytics engine 260 loads one or more recipes from the recipe library 170, as shown in 338. The analytics engine 260 determines, based on the request, the recipes to be loaded, and the order in which they are loaded. Recipes can be segments of code implementing an algorithm or operation or machine learning models. Examples of a recipe include changing the color or resolution of an image. There is no limit to the number or types of recipes that may be used, also extending the scope of the orchestrator software 100 well beyond radiology and medicine.

At operation 340, the analytics engine 260 executes the one or more recipes in accordance with the recipe. At operation 342, the analytics engine 260 sends results to the kernel 110. At operation 314, the kernel 110 updates the IDS with analytics results.

In some embodiments, multiple analysis recipes can be run simultaneously in the analytics engine 260. Optionally, the method 300 includes operations 346, 348, 352, 354, and 356, which are similar to operations 334, 336, 340, 342, and 344, respectively, except for a second analysis recipe. Optionally, at operation 348, the analytics engine 260 loads the filter library 170, as shown in 350, which is similar to 338.

At operation 358, either in response to determining that no analysis from analytics engine 260 is required at operation 332, or in response to updating the JDS in operation 344 or operation 356, the inference engine 190 determines whether the inference engine 190 has executed a last step of the ruleset. In the case that the inference engine 190 determines that no analysis is from analytics engine 260 is required the inference engine 190 can use a decision tree and inputs provided in the request or the JDS to determine the results. In response to determining that the inference engine 190 has not executed the last step of the ruleset, the method 300 returns to operation 324. In response to determining that the inference engine 190 has executed the last step of the ruleset, at operation 360, the inference engine 190 interprets the results.

At operation 362, the inference engine 190 determines whether a conclusion is reached. The conclusion can be one of: the inference engine 190 has converged on an answer to the question or request of operation 302, or the inference engine 190 determines that the inference engine 190 cannot converge on an answer to the question or request of operation 302. In response to determining that a conclusion is not reached (e.g., more iterations are needed), the method 300 returns to operation 324. In response to determining that a conclusion is reached, either via a convergence or a failure to converge, at operation 364, the inference engine 190 sends the results to the kernel 110.

At operation 366, the kernel 110 updates the JDS. In some embodiments, the kernel 110 records the results in the data mesh 180. At operation 368, the codex 130 translates the results. At operation 370, the console 120 provides the results to the user. Examples of results include raw data or clinical information; all data for a time point and body site; data for body site across time points; single voxel in a MIBA file, biomap, digital twin, 3D file, or graph database; data for a single voxel in a MIBA file, biomap, digital twin, 3D file, or graph database, etc.

FIG. 3 shows the basic flow can be invoked repeatedly for different body sites, time points or patients, and the rulesets will continue to run in parallel updating the databases: graph, MIBA file as appropriate when they complete. Within any given ruleset, multiple analytics recipes may be run in parallel with the results updated to the graph, MIBA file as each recipe completes.

In some embodiments, the MIBA File/Digital Twin/Precision Biomap Generator MIBA File/Digital Twin/Precision Biomap generator 240 is a batch process that is triggered by a referral order created in an EHR 150 or an update to any part of the EHR for a patient that has already been loaded into data mesh 180 or by some other user command. Based on information in the referral order or update, the MIBA File/Digital Twin/Precision Biomap Generator MIBA File/Digital Twin/Precision Biomap 240 can gather patient data from the EHR 150, images from the PACS 160, and real-time information from other sources 210 and build a graph or MIBA file using feature labels on the images for mapping and/or standard registrations, or, embodiment of the data mesh 180. If a biomap already exists for the patient, any new information can be added and modified information updated in the existing biomap. The patient can be added to the patient queue 250 to indicate the patient data is ready for expert user review.

In some embodiments, data across sessions is collected and analyzed by the decision intelligence analytics engine 220. Upon request, a user may view the collected data on a decision intelligence console UI 230. The data collected and displayed can be configurable by customer installation.

The interface between the kernel 110 and the console 120 (e.g., via inbound codex 130a) can transmit commands, requests for information, queries, and non-graphical annotations sent from the console 120 to the kernel 110. Depending on the context and the content, the kernel 110 may forward the command, request, query, or annotation to another component. A function of the kernel 110 can be to intelligently direct traffic and manage the state of sessions with the consoles 120 connected to the patient. The inbound codex 130a can translate the data from the mode used on the console 120 to that used by the kernel 110. The inbound codex 130a can be an asynchronous interface. Content transmitted by the inbound codex 130a can include commands, requests, queries, non-graphical annotations sent from the console 120 to the kernel 110. Most of these messages can be forwarded to other components based on context, the state of the session, and the nature of the contents.

The interface between the kernel 110 and the console 120 (e.g., via inbound codex 130b) can transmit data sent from the kernel 110 to the console 120 except images. The codex pairs serve as a means to translate the content from the mode used internally by the kernel 110 and that used by the user via the console 120. Possible modes include voice, text, or visualizations that do not require rendering (these go through the 3D view pipeline 140). The outbound codex 130b can be an asynchronous interface. Content transmitted by the inbound codex 130a can include data to be presented on the console 120. The console 120 can decide how to present the data based on context and the mode chosen by the user. The console 120 can invoke the appropriate codex 130 to perform any required mode translation.

The interface between the kernel 110 and the 3D view pipeline 140 can transmit data required to render 3D datacubes, raw images, and segmented 2D or 3D ROI on the console 120. Due to the size of some images, the data transmitted may be adjusted to fit within the constraints of the network and/or the device hosting the console, such that only parts of an image are downloaded or video streaming techniques are used. The interface between the kernel 110 and the 3D view pipeline 140 can be a synchronous interface. Content transmitted by the interface between the kernel 110 and the 3D view pipeline 140 can include data is transmitted as 3D or 4D numerical arrays which may be raw or rendered images as befitting the context.

The interface between the console 120 and the 3D view pipeline 140 can transmit annotations of regions of interest (ROI) and specific segmented ROI created by the user on the console 120 used by the user to define the IDS. The interface between the console 120 and the 3D view pipeline 140 can be an asynchronous interface. Content transmitted by the interface between the console 120 and the 3D view pipeline 140 can include data is transmitted as 3D or 4D numerical arrays.

Pre-rendered images in a “smartview” engine 270 can be drawn by the 3D view pipeline 140 from the library 270 to display on the console 120 to represent anatomical structures for which there is data in the current data mesh 180. The interface between the 3D view pipeline 140 and the smart view library 270 can be an asynchronous interface. Content transmitted by the interface between the 3D view pipeline 140 and the smart view engine 270 can include images, including pre-rendered library images, are transmitted as 3D or 4D numerical arrays.

Images and annotations, in the form of a 3D or 4D numerical array, can be passed from the kernel 110 into the analytics engine 260 along with a selection of the processing to be done. Results of that processing, in the form of a modified 3D or 4D numerical array and specific findings that are discovered, are passed back to the kernel 110 for routing to the next step. The processing done may involve a sequence of networks 170. The interface between the kernel 110 and the image analytics engine 260 can be an asynchronous interface. Content transmitted by the interface between the kernel 110 and the image analytics engine 260 can include 3D or 4D numerical arrays, plus command or some other indication of the processing sequence to use, and non-image findings.

The analytics engine 260 can decide which filters 170 to use and in which order. Each filter 170 can be passed in the 3D or 4D numerical array. In some embodiments, the 3D or 4D numerical array is modified by the filter. The modified array can be passed back to the image analytics engine 260 which may pass it to another filter or return it to the kernel 110. The interface between the image analytics engine 260 and the image filters 170 can be a synchronous interface. Content transmitted by the interface between the image analytics engine 260 and the image filters 170 can include 3D or 4D numerical array, plus a binary value to indicate whether the filter found the indication it was built to find or a successful operation for image modification filters (such as edge detection, noise reduction, or probability of a specific biomarker question, such as “is this cancer?”).

The kernel 110 may gather findings from the image analytics engine 260 and the user via the console 110 and presents the findings to the inference engine 190. The interface between the kernel 110 and the inference engine 190 can be an asynchronous interface. Findings can take the form of identified features found on images identified by either the image analytics engine 260 or the user via the console 120. The form can be a list or dictionary. The dictionary can be used when there is descriptive data about a finding.

The patient queue 250 may be accessed by the kernel 110 via a Uniform Resource Identifier (URI) and either a GET or DELETE command. The kernel 110 can pull a patient from the patient queue 250, for example, using a GET request based on input from the user via the console 120. Patients pulled from the patient queue 250 can be removed from by the kernel 110, for example, using a DELETE command. This can have the effect or removing the patient from the queue but may have no effect on any other data. The interface between kernel 110 and the patient queue 250 can be an asynchronous interface.

The kernel 110 may make specific requests for data from an EHR 150 or may update the EHR 150 with a new or modified report. The interface between kernel 110 and the EHR 150 can be an asynchronous interface. Communication between the kernel 110 and the EHR 150 may be via Fast Healthcare Interoperability Resources (FHIR) packaged as block of JavaScript Object Notation (JSON) data.

The kernel 110 may need to request a specific image from a PACS 160. Such requests can be originated by the user via the console 120. The interface between kernel 110 and the PACS 160 can be an asynchronous interface. Image requests are made via C-Move or C-Copy commands as defined by the Digital Imaging and Communications in Medicine (DICOM) standard.

The kernel 110 may request data from other (external) sources 210 such as a connected device. These requests can be originated by the user via the console 120, but may be originated directly to provide data required for an operation or analysis. The interface between kernel 110 and the other sources 210 can be an asynchronous interface. Data can be transmitted as JSON data.

The kernel 110 can pull information from the data mesh 180 as needed to pass to the console 120 for display, or to other components for their operations. The kernel 110 may make updates to the information in the data mesh 180. The interface between the kernel 110 and the data mesh 180 can be an asynchronous interface. The data mesh 180 can be accessed via a URI and one of the commands including GET, POST, PUT, or DELETE.

The MIBA File/Digital Twin/Precision Biomap Generator MIBA File/Digital Twin/Precision Biomap generator 240 can gather data from the EHR 150 triggered by an event in the EHR 150. The triggering event can be an update of data for an existing patient. The triggering event can be the addition of a new referral order for a radiology review. Triggering events can be configured into the MIBA File/Digital Twin/Precision Biomap Generator MIBA File/Digital Twin/Precision Biomap generator 240. The interface between the MIBA File/Digital Twin/Precision Biomap Generator MIBA File/Digital Twin/Precision Biomap generator 240 and the EHR 150 can be an asynchronous interface. Data can be in the form of FHIR packaged as JSON data.

The process of gathering patient data for review by the user can include pulling a standard set of images from the PACS 160. The specific images in a standard set depend on the anatomical structure under review, the nature of the suspected condition, and what is available in the PACS 160 and can be defined using a rule-set evaluated by the inference engine 190. The interface between the MIBA File/Digital Twin/Precision Biomap Generator MIBA File/Digital Twin/Precision Biomap generator 240 and the PACS 160 can be an asynchronous interface. Image requests can be made via C-Move or C-Copy commands as defined by the DICOM standard.

Data required from external sources may be in the EHR 150, but direct access by the MIBA File/Digital Twin/Precision Biomap Generator MIBA File/Digital Twin/Precision Biomap 240 may be required. The interface between the MIBA File/Digital Twin/Precision Biomap Generator MIBA File/Digital Twin/Precision Biomap generator 240 and the other sources 210 can be an asynchronous interface. Data can be transmitted as JSON blocks.

A patient data mesh 180 for the patient can be created by the MIBA File/Digital Twin/Precision Biomap Generator MINA File/Digital Twin/Precision Biomap generator 240 and added to the set of data mesh 180. If a data mesh 180 exists for a patient, the generator 240 can update the existing patient data mesh 180. The interface between the MIBA File/Digital Twin/Precision Biomap Generator MIBA File/Digital Twin/Precision Biomap generator 240 and the patient data mesh 180 can be an asynchronous interface. The API for the biomap can include a URI identifying the patient and a GET, POST, PUT, or DELETE command. Data can be transmitted as JSON blocks.

Once a patient's data has been collected and the patient data mesh 180 updated, the patient can be added to the patient queue 250. The interface between the MIBA File/Digital Twin/Precision Biomap Generator MIBA File/Digital Twin/Precision Biomap generator 240 and the patient queue 250 can be a synchronous interface. The patient queue 250 can be updated via a PUT command through the queue's API.

Data about decisions made by both the user and the system can be monitored for multiple effectiveness measures. The decision analytics engine 220 can maintain the set and definitions of metrics used to assess decision effectiveness and make requests for specific data of the kernel 110. The interface between the kernel 110 and the decision analytics engine 220 can be a synchronous interface. Data requested by the decision analytics engine 220 can collected and provided by the kernel 110. The data that traverses this interface can depend on the set and nature of the metrics collected.

Data collected and analyzed by the decision analytics engine 220 may be rendered and sent to the DI console 230 for display. The interface between the decision analytics engine 220 and the DI console 230 can be an asynchronous interface. Data can be transmitted as JSON blocks, including instructions about how to construct a visualization to present the data on the DI console 230.

For UC 1, the user requests to view data through the console 120, which communicates the request to the kernel 110 via the codex 130. The kernel 110 loads the data from the patient data mesh 180, accessing all time points available, and returns the data to the console 120 via the codex 130. If the data is imaging data or other 3D data, then the kernel 110 can use the 3D view pipeline to access the imaging data or other 3D data.

For UC 2, the user request selects an anatomical location and request data through the console 130. The console communicates the location and request to the kernel 110 via the codex 130. If an appropriate joint data space exists, the kernel 110 returns the information in the JDS to the console 120 via the codex 130. If the JDS does not exist, the kernel 110 allocates one and loads it with data from the patient data mesh 180, which is then transmitted to the console 120 via the codex 130.

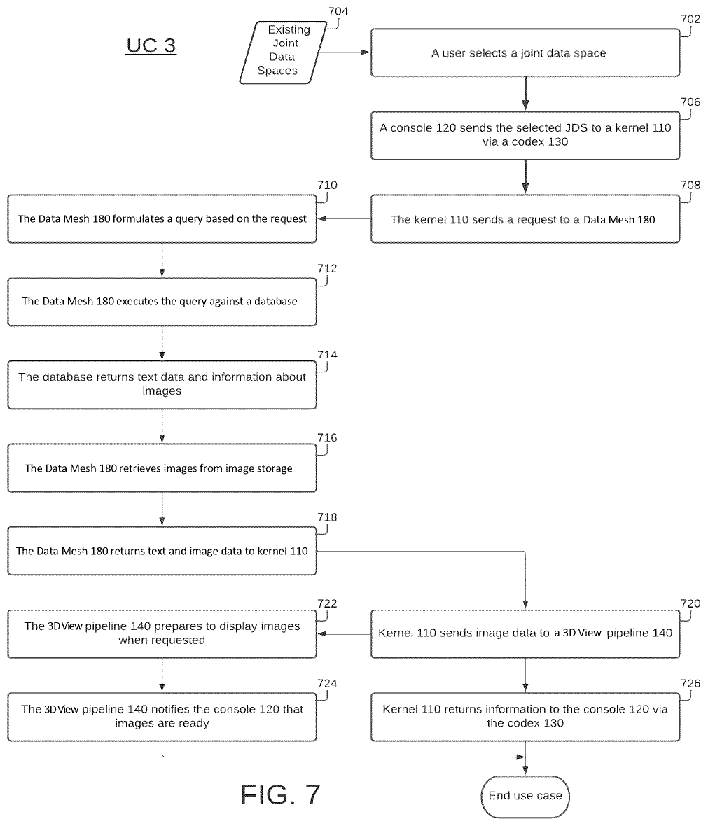

FIG. 7 illustrates a use case UC 3, in accordance with some embodiments. At operation 702, a user selects a joint data space 704 by interacting with a user console. The user selects one of the existing joint data spaces through the console 120. At operation 706, the console transmits the selected JDS to the kernel 110 via the codex 130.

At operation 708, the kernel 110 sends a request to a data mesh 180. At operation 710, the data mesh 180 formulates a query based on the request. At operation 712, the data mesh 180 executes the query against a database (e.g., the graph database or other database). At operation 714, the database returns text data and information about the images. At operation 716, the data mesh 180 retrieves images from image storage. At operation 718, the data mesh 180 returns text and image data to kernel 110.

At operation 720, the kernel 110 sends image data to a 3D view pipeline 140. At operation 722, the 3D view pipeline 140 prepares to display images when requested. At operation 724, the 3D view pipeline 140 notifies the console 120 that the images are ready. At operation 726, the kernel 110 returns information to the console 120 via the codex 130. In some embodiments, the operation 726 occurs in parallel with the operations 722 and 724. In some embodiments, the kernel generates a list of anatomical locations in the JDS and transmits the list and the JDS to the console 120 via the codex 130.

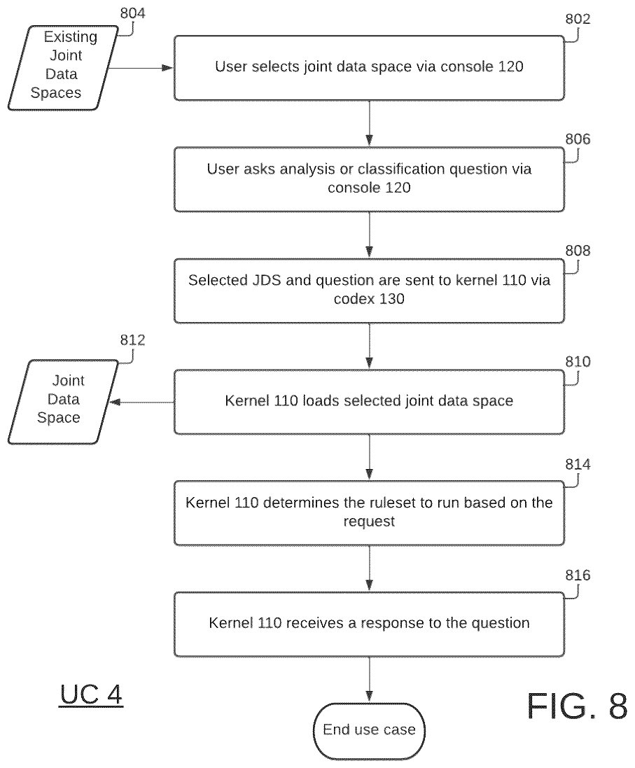

FIG. 8 illustrates a use case UC 4, in accordance with some embodiments. At operation 802, the user selects one of the existing joint data spaces 804 through the console 120. At operation 806, the user also asks a question through the console 120. At operation 808, the console 120 sends the selected JDS and question to the kernel 110 via the codex 130. At operation 810, the kernel 110 loads the JDS 812. At operation 814, the kernel 110 determines the ruleset to run based on the question. At operation 816, the kernel 110 receives a response to the question, e.g., from one or more components executing the ruleset.

For UC 5, the user first executes UC4. The inference engine 190 loads the selected ruleset from the ruleset library 200, then executes it. If additional information is required, the inference engine 190 requests the information from the kernel 110, which collects from the patient data mesh 180 and returns it to the inference engine 190. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the JDS. The kernel 110 notifies the ruleset that the results are available. The ruleset runs until it completes or cannot converge to a solution as defined in the ruleset. The inference engine notifies the kernel 110 and returns the results, which are loaded into the JDS. The kernel 110 then notifies the console 120 via the codex 130 that results are ready.

For UC 6, the user requests to see all running inference processes related to the current joint data space through the console 120. The set of processes are shown, along with their status (running, paused). The user selects a process and a new status. The user may pause or stop a running process, or may resume or stop a paused process. The selected process and new status are sent to the kernel 110 via the codex 130. The kernel sends the command to the selected process via the inference engine 190. The inference engine 190 executes the command and returns the new status to the kernel 110. The kernel 110 updates the joint data space and informs the console 120 via the codex 130.

For UC 7, the user requests an analysis be performed on a patient cohort based on the current joint data structure through the console 120. The console sends the request to the kernel 110 via the codex 130. The kernel 110 instructs the inference engine 190 to build a patient cohort based on the current IDS. The inference engine 190 loads and executes the ruleset from the ruleset library 200, which requests the IDS. The ruleset may search through other patient data mesh (180), query the EHR 150 directly, or search through external health record banks for patients that meet the criteria for the cohort. As patients are found, and consents are validated, they are added to the cohort. When complete, the cohort is returned to the kernel 110 which then directs the inference engine 190 to perform the requested analysis. The inference engine 190 loads and executes the ruleset from the ruleset library 200. The ruleset requests the cohort from the kernel 110 and iterates through each patient data mesh 180 in the cohort. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the IDS. The kernel 110 notifies the ruleset that the results are available. The ruleset runs until it has processed all patients in the cohort. The inference engine notifies the kernel 110 and returns the results, which are loaded into the IDS. The kernel 110 then notifies the console 120 via the codex 130 that results are ready.

For UC 8, the user first executes UC4. The inference engine 190 loads the selected ruleset from the ruleset library 200, then executes it. If additional information is required, the inference engine 190 requests the information from the kernel 110, which collects from the patient data mesh 180 and returns it to the inference engine 190. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the IDS. The kernel 110 notifies the ruleset that the results are available. The ruleset runs until it completes or cannot converge to a solution as defined in the ruleset. The inference engine notifies the kernel 110 and returns the results, which are loaded into the IDS. The kernel 110 then notifies the console 120 via the codex 130 that results are ready.

For UC 9, the user requests an analysis or patient cohort be prepared for later use via the console 120. The request is sent to the kernel 110 via the codex 130. If the request is for an analysis, the kernel 110 loads the recipe from the filter library 170 into the joint data space and notifies the console 120 via the codex 130 that results are ready. If the request is for a patient cohort, the kernel 110 instructs the inference engine 190 to build a patient cohort based on the current IDS. The inference engine 190 loads and executes the ruleset from the ruleset library 200, which requests the IDS. The ruleset may search through other patient data mesh (180), query the EHR 150 directly, or search through external health record banks for patients that meet the criteria for the cohort. As patients are found, and consents are validated, they are added to the cohort. When complete, the cohort is returned to the kernel 110 which then updates the joint data space and informs the console 120 via the codex 130 that the information is ready.

For UC 10, the user selects one of the existing joint data spaces through the console 120. The console 120 sends the requested IDS to the kernel 110 via the codex 130. The kernel 110 loads the IDS and updates it with new information from the patient data mesh 180, then informs the console 120 via the codex 130 that the information is ready.

For UC 11, logging of actions taken by the user can be turned on or off. When turned on, changes to the patient data mesh are recorded and saved. When turned off, changes are not saved beyond the end of the session.

For UC 12, the user first executes UC 4. The inference engine 190 loads the selected ruleset from the ruleset library 200, then executes it. If additional information is required, the inference engine 190 requests the information from the kernel 110, which collects from the patient data mesh 180 and returns it to the inference engine 190. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the IDS. The kernel 110 notifies the ruleset that the results are available. The ruleset runs until it completes or cannot converge to a solution as defined in the ruleset. The inference engine notifies the kernel 110 and returns the results, which are loaded into the IDS. The kernel 110 then notifies the console 120 via the codex 130 that results are ready.

For UC 13, the user selects one of the existing joint data spaces via the console 120, then invites other users to view the IDS, also via the console 120. The console 120 notifies the kernel 110 via the codex 130 of the invitations and the selected IDS. The kernel 110 issues the invitations. As responses are received, the kernel 110 connects each new user to the IDS.

For UC 14, the user requests an analysis be performed on a patient cohort based on the current joint data structure through the console 120. The console sends the request to the kernel 110 via the codex 130. The kernel 110 instructs the inference engine 190 to build a patient cohort based on the current IDS. The inference engine 190 loads and executes the ruleset from the ruleset library 200, which requests the IDS. The ruleset may search through other patient data mesh (180), query the EHR 150 directly, or search through external health record banks for patients that meet the criteria for the cohort. As patients are found, and consents are validated, they are added to the cohort. When complete, the cohort is returned to the kernel 110 which then directs the inference engine 190 to perform the requested analysis. The inference engine 190 loads and executes the ruleset from the ruleset library 200. The ruleset requests the cohort from the kernel 110 and iterates through each patient data mesh 180 in the cohort. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the IDS. The kernel 110 notifies the ruleset that the results are available. The ruleset runs until it has processed all patients in the cohort. The inference engine notifies the kernel 110 and returns the results, which are loaded into the IDS. The kernel 110 then notifies the console 120 via the codex 130 that results are ready.

For UC 15, the user requests an analysis be performed on a patient cohort based on the current joint data structure through the console 120. The console sends the request to the kernel 110 via the codex 130. The kernel 110 instructs the inference engine 190 to build a patient cohort based on the current IDS. The inference engine 190 loads and executes the ruleset from the ruleset library 200, which requests the IDS. The ruleset may search through other patient data mesh (180), query the EHR 150 directly, or search through external health record banks for patients that meet the criteria for the cohort. As patients are found, and consents are validated, they are added to the cohort. When complete, the cohort is returned to the kernel 110 which then directs the inference engine 190 to perform the requested analysis. The inference engine 190 loads and executes the ruleset from the ruleset library 200. The ruleset requests the cohort from the kernel 110 and iterates through each patient data mesh 180 in the cohort. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the IDS. The kernel 110 notifies the ruleset that the results are available. The ruleset runs until it has processed all patients in the cohort. The inference engine notifies the kernel 110 and returns the results, which are loaded into the IDS. The kernel 110 then notifies the console 120 via the codex 130 that results are ready.

For UC 16, the user requests an analysis be performed over the entire patient journey based on the current joint data space via the console 120. The console notifies the kernel 110 via the codex 130 of the request. The inference engine 190 loads and executes the requested ruleset from the ruleset library 200. The ruleset requests patient history from the patient data mesh 180 via the kernel 110, which retrieves the information and informs the inference engine 190 that the information is ready. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the JDS. The kernel 110 notifies the ruleset that the results are available. The ruleset runs until it completes or cannot converge to a solution as defined in the ruleset. The inference engine notifies the kernel 110 and returns the results, which are loaded into the JDS. The kernel 110 then notifies the console 120 via the codex 130 that results are ready.

For UC 17, the user requests an analysis be performed on a patient cohort based on the current joint data structure through the console 120. The console sends the request to the kernel 110 via the codex 130. The kernel 110 instructs the inference engine 190 to build a patient cohort based on the current JDS. The inference engine 190 loads and executes the ruleset from the ruleset library 200, which requests the JDS. The ruleset may search through other patient biomaps (180), query the EHR 150 directly, or search through external health record banks for patients that meet the criteria for the cohort. As patients are found, and consents are validated, they are added to the cohort. When complete, the cohort is returned to the kernel 110 which then directs the inference engine 190 to perform the requested analysis. The inference engine 190 loads and executes the ruleset from the ruleset library 200. The ruleset requests the cohort from the kernel 110 and iterates through each patient data mesh 180 in the cohort. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the JDS. The kernel 110 notifies the ruleset that the results are available. The ruleset runs until it has processed all patients in the cohort. The inference engine notifies the kernel 110 and returns the results, which are loaded into the JDS. The kernel 110 then notifies the console 120 via the codex 130 that results are ready.

For UC 18, the user first executes UC 4. The kernel 110 directs the inference engine 190 to execute the selected ruleset. The inference engine 190 loads and executes the ruleset from the ruleset library 200. If additional information is required, the inference engine 190 requests the information from the kernel 110, which collects from the patient data mesh 180 and returns it to the inference engine 190. If analytics are required by the ruleset, the inference engine notifies the kernel 110 which instructs the analytics engine 260 to run the specified recipe. The analytics engine loads the recipe from the filter library 170 and executes it, possibly requesting addition information from the kernel 110. After completion, the analytics engine 260 notifies the kernel 110 and returns the results, which are loaded into the JDS. The kernel 110 notifies the ruleset that the results are available. The ruleset runs until it completes or cannot converge to a solution as defined in the ruleset. The inference engine notifies the kernel 110 and returns the results, which are loaded into the JDS. The kernel 110 then notifies the console 120 via the codex 130 that results are ready.

In some embodiments, as follow up occurs, the patient's information is kept up to date in the EHR 150 which can trigger an update of the patient data mesh 180 by the MIBA File/Digital Twin/Precision Biomap Generator MIBA File/Digital Twin/Precision Biomap generator 240. The use case described by the method 200 may be repeated many times over the course of treatment.

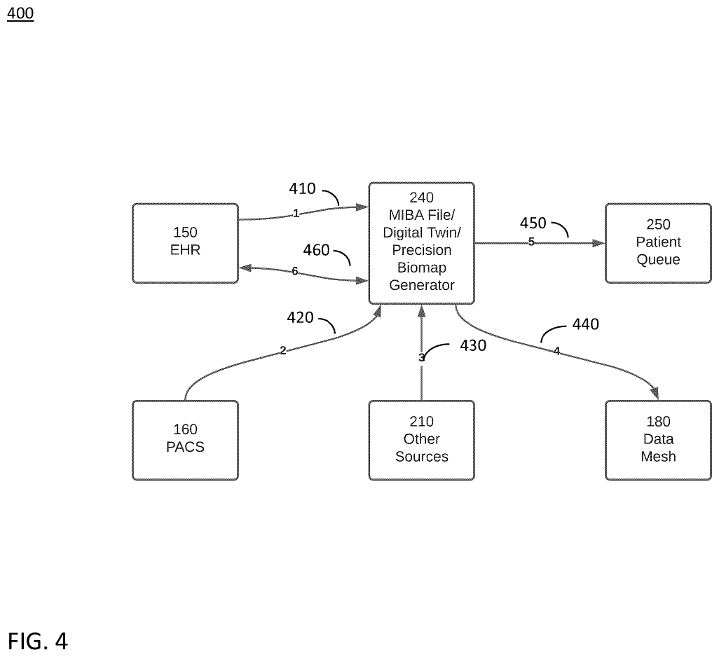

FIG. 4 is a functional flow block diagram of a method 400 for creating a data mesh, in accordance with an illustrative embodiment and prior MIBA and precision biomap patents. The method 400 may be implemented using, or performed by one or more of the operating software 100, one or more components of the operating software 100, or a processor associated with the operating software 100 or one or more of the components. Additional, fewer, or different operations may be performed in the method 400 depending on the embodiment. Additionally, or alternatively, two or more of the operations or embodiments of the method 400 may be performed in parallel. Operations or embodiments of the method 400 may be combined with one or more operations or embodiments of one or more of the methods 200-300.

Some embodiments may be triggered by an event in the EHR 150 detected by the operating software 100. At operation 410, in some embodiments, one of several tracked events occurs in the EHR 150 and the interface between the EHR 150 and the MIBA File/Digital Twin/Precision Biomap Generator MIBA File/Digital Twin/Precision Biomap generator 240 triggers a MIBA File/Digital Twin/Precision Biomap Generator MIBA File/Digital Twin/Precision Biomap generator 240 batch process. At operation 420, in some embodiments, the MIBA File/Digital Twin/Precision Biomap Generator MIBA File/Digital Twin/Precision Biomap generator 240 queries the EHR 150 for information on the patient that triggered the event and loads the data via the interface (e.g., the third party interfaces).

At operation 430, in some embodiments, new images for the patient are queried and loaded from the PACS 160. At operation 440, in some embodiments, the MIBA File/Digital Twin/Precision Biomap Generator MI BA File/Digital Twin/Precision Biomap generator 240 checks to see if a patient data mesh 180 exists for the patient that triggered the event. If one exists, the patient data mesh 180 for the patient can be updated with the new information under user command. If one does not exist, one can be created. At operation 450, in some embodiments, the patient is added to the patient queue 250 or the patient's information is updated if the patient is already in the queue.

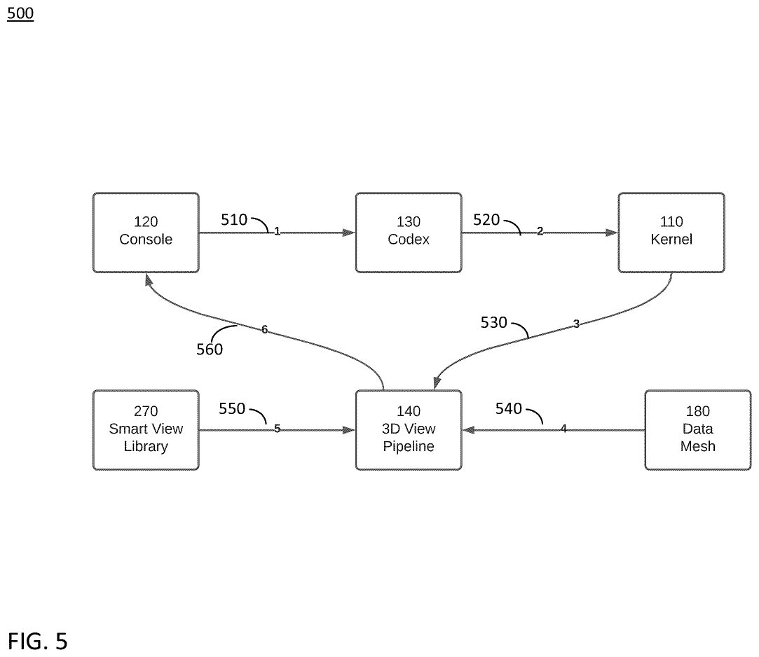

FIG. 5 is a functional flow block diagram of a method 500 for viewing a specific image, in accordance with an illustrative embodiment. The method 500 may be implemented using, or performed by one or more of the operating software 100, one or more components of the operating software 100, or a processor associated with the operating software 100 or one or more of the components. Additional, fewer, or different operations may be performed in the method 500 depending on the embodiment. Additionally, or alternatively, two or more of the operations or embodiments of the method 500 may be performed in parallel. Operations or embodiments of the method 500 may be combined with one or more operations or embodiments of one or more of the methods 200-400.

In some embodiments, a user may access the operating software 100 wanting to view a specific image and not go through the use case to select the next patient. In some embodiments, the specific image is a voxel or a portion of a voxel. At operation 510, in some embodiments, a user requests a specific image for a specific patient, such as “the latest FLAIR MRI for patient X,” via the console 120 through the codex 130. The request may be transformed by the codex 130 to put it in the form needed by the backend. At operation 520, in some embodiments, the codex 130 can forward the transformed request to the kernel 110.

At operation 530, in some embodiments, the image request is sent by the kernel 110 to the 3D view pipeline 140 along with information about where the image pixel data is located. At operation 540, in some embodiments, the 3D view pipeline 140 queries the patient data mesh 180, the MIBA file, the 3D precision biomap, or the digital twin for specific information about the image and then loads the pixel data. At operation 550, in some embodiments, the 3D view pipeline 140 uses resources in the smart view library to process and format the image for display. At operation 560, in some embodiments, the 3D view pipeline 140 sends part of the image in each of three planes to the console 120 for display. The 3D view pipeline 140 can manage the amount of data sent to the console 120 to prevent an overload condition.

In summary, FIG. 5 illustrates how the kernel 110 can invoke the 3D view pipeline 140 which can then work directly with the console 120 to manage the transmission and display of an image. The connection between the console 120 and 3D view pipeline 140 may be synchronous because of the close coordination that may be required.

According to one embodiment of the disclosure, orchestrator 100 performs specific operations by a processor executing one or more sequences of one or more instructions contained in system memory. Such instructions may be read into system memory from another computer readable/usable medium, such as static storage device or disk drive. In alternative embodiments, hard-wired circuitry may be used in place of or in combination with software instructions. Thus, particular embodiments are not limited to any specific combination of hardware circuitry and/or software.

The term “computer readable medium” or “computer usable medium” as used herein refers to any medium that participates in providing instructions to processor for execution. Such a medium may take many forms, including but not limited to, nonvolatile media and volatile media. Non-volatile media includes, for example, optical or magnetic disks, such as disk drive. Volatile media includes dynamic memory, such as system memory. Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

In particular embodiments, the orchestrator 100 may be hosted on a single computer system. According to other embodiments, the orchestrator 100 may be distributed over two or more computer systems coupled by communication link (e.g., LAN, PTSN, WAN, or other public/private, wired/wireless network) may perform the sequence of instructions in coordination with one another. The two or more computer systems may be in a same geographic region or in different geographic regions. At least one computer system may be a cloud such cloud computing system (such as a public cloud, a private cloud, a hybrid cloud, a multicloud, or a co-location facility), a private data center, one or more physical servers, virtual machines, or containers of an entity or customer. In some embodiments, a portion of the orchestrator 100 is in a cloud computing system and another portion is in a private data center. For example, the kernel 110 can reside in a private data center and the data mesh 180 can reside in a separate cloud computing system (e.g., as part of a Health Record Bank). Other combinations of computing systems are within the scope of the present disclosure.

It is to be understood that any examples used herein are simply for purposes of explanation and are not intended to be limiting in any way.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to disclosures containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the disclosure be defined by the claims appended hereto and their equivalents.

What is claimed is:

1. A method for orchestrating a human-information interaction (HII) system including:
creating, by a kernel, a joint data space (JDS) based on one or more user selections;
receiving, by the kernel, a user request;
selecting, by the kernel, a ruleset based on the user request;
determining, by the kernel, components for executing the request in accordance with the ruleset;
sending, by the kernel, portions of the ruleset to corresponding ones of the components;
receiving, by the kernel, results from the components;
determining, by the kernel, that a user approves updating a data mesh with the results; and
recording, by the kernel, the results in the data mesh.

2. The method of claim 1, wherein the components include an inference engine that determines whether analysis is to be performed in executing the ruleset.

3. The method of claim 1, wherein the components include an analytics engine that executes filters to generate at least a portion of the results.

4. The method of claim 1, wherein the kernel determines the ruleset based on one or more of:
a body site that is selected by the user;
a current decision stage; or
a type of the user request.

5. The method of claim 4, wherein the type of the user request includes one of:
categorization;
filtering; or
prediction.

6. The method of claim 1, further comprising determining, by the kernel, a current state of a joint data space (JDS).

7. The method of claim 1, wherein the JDS points, using an identification (ID) tag, to a node in the data mesh.

8. The method of claim 1, wherein the data mesh points, using an identification (ID) tag, to one or more analytics databases.

9. The method of claim 1, wherein the components include an intelligence agent that receive inputs and takes actions responsive to the received inputs such that the actions evolve over time with operation of the HII system.

10. A system comprising:
a joint data space (JDS) created based on one or more user selections;
a kernel configured to receive a user request, wherein the kernel is further configured to:
select a ruleset based on the user request;
determine components for executing the request in accordance with the ruleset;
send portions of the ruleset to corresponding ones of the components;
receive results from the components;
determine that a user approves updating a data mesh with the results; and
record the results in the data mesh.

11. The system of claim 10, further an intelligence agent that receive inputs and takes actions responsive to the received inputs such that the actions evolve over time with operation of the HII system.

12. The system of claim 10, further comprising an analytics engine that executes filters to generate at least a portion of the results.

13. The system of claim 1, wherein the kernel is further configured to determine the ruleset based on one or more of:
a body site that is selected by the user;
a current decision stage; or
a type of the user request.

14. The system of claim 13, wherein the type of the user request includes one of:
categorization;
filtering; or
prediction.

15. The system of claim 10, wherein the kernel is further configured to determine a current state of a joint data space (JDS).

16. The system of claim 10, wherein the JDS comprises an identification tag that points to a node in the data mesh.

17. The system of claim 10, wherein the data mesh comprises an identification tag that points to one or more analytics databases.

18. The system of claim 10, further comprising an inference engine that determines whether analysis is to be performed in executing the ruleset.

19. A non-transitory computer-readable medium having instructed stored thereon, that upon execution, cause a computing device to perform operations comprising:
creating a joint data space (JDS) based on one or more user selections;
receiving a user request;
selecting a ruleset based on the user request;
determining components for executing the request in accordance with the ruleset;
sending portions of the ruleset to corresponding ones of the components;
receiving results from the components;
determining that a user approves updating a data mesh with the results; and
recording the results in the data mesh.
